(19) United States
(12) Patent Application Publication
(10) Pub. No.: US 2022/0273727 A1
(43) Pub. Date: Sep. 1, 2022

(54) HOLLOW THREE-DIMENSIONAL UNIT MADE FROM RETINAL TISSUE AND USE THEREOF IN THE TREATMENT OF RETINOPATHIES

(71) Applicant: TREEFROG THERAPEUTICS, PESSAC (FR)

(72) Inventors: Maxime FEYEUX, TALENCE (FR); Kevin ALESSANDRI, BORDEAUX (FR)

(21) Appl. No.: 17/634,794

(22) PCT Filed: Aug. 12, 2020
(86) PCT No.: PCT/EP2020/072567

§ 371 Date: Feb. 11, 2022

(30) Foreign Application Priority Data
Aug. 12, 2019 (FR) FR1909155

(51) Int. Cl. A61K 35/30; A61L 27/20; A61L 27/38; A61L 27/52; A61P 27/02

(52) U.S. Cl. CPC A61K 35/30; A61L 27/20; A61L 27/3813; A61L 27/3834; A61L 27/52; A61P 27/02; A61F 2/14

(57) ABSTRACT

The invention relates to three-dimensional tissue units which are hollow and which comprise, when organized about an internal opening, at least one layer of living human retinal pigment epithelium cells which are differentiated, the basal face of each cell pointing outwards and the apical face pointing towards the internal opening. The invention also relates to these tissue units for use in the treatment of retinopathies, and to a method for preparing these tissue units and an implantation kit.

### CROSS-REFERENCE TO RELATED APPLICATIONS

This application is the U.S. national stage application of PCT/EP2020/072567 with the international filing date of Aug. 12, 2020 and claiming the benefit of priority from French patent application FR 1909155 filed Aug. 12, 2019, the entire disclosure of these applications is herein incorporated by reference.

### TECHNICAL FIELD

The present invention relates to the treatment of retinal diseases, in particular by the use of specific tissue units comprising at least one retinal pigment epithelium. The invention also relates to a method for preparing these tissue units and to kits for implanting these tissue units in the eye to perform transplants of all or part of the retina.

### BACKGROUND

Retinal diseases, or retinopathies, are one of the major causes of visual impairment in the world. The retina, which lines the back of the eye, is made up in particular of pigment epithelial cells and nerve cells that receive light. These nerve cells translate the light into electrical signals that travel to the brain via the optic nerve. When the cells of the retina, in particular of the retinal pigment epithelium, degenerate or stop functioning, blind areas of the visual field appear.

Retinopathies can have various origins: in particular, they can be related to aging, such as age-related macular degeneration (AMD), can be hereditary, such as retinopathy pigmentosa or retinal dystrophy, can be related to a trauma, such as solar retinopathy, or can derive from another pathology, such as diabetic retinopathy or hypertensive retinopathy.

AMD is the leading cause of visual impairment in the elderly. This pathology is due to the damage of the macula, the central area of the retina, which transmits most of the visual information to the brain. It results in the appearance of a blind spot in the center of the visual field. AMD can present itself in two forms:
a dry or atrophic form: it is characterized by the progressive disappearance of the cells of the macula. It therefore develops slowly and represents the most common form of AMD;
a wet or exudative form: it is characterized by the formation of abnormal blood vessels under the retina which lead in particular to its detachment.

There is currently no treatment for the dry form of AMD. The only way is to delay it by taking food supplements (vitamins C and E and antioxidant minerals). For a few years now, for wet AMD, if the disease is not at a too advanced stage, an injection into the eye of a drug that blocks the proliferation of the vessels can allow an improvement of the disease, but this treatment requires a monthly injection and does not make it possible to obtain entirely satisfactory results.

Retinopathy pigmentosa is a genetic disease that can be inherited. It is characterized by a degeneration of the cells of the retina linked to the mutation of one or more genes. The evolution of the disease is slow until it leads to blindness; there is currently no treatment.

Diabetic retinopathy is a retinal damage occurring in the context of diabetes. It is related to the excessive concentration of sugar in the small blood vessels of the retina, which leads to their degradation. The lack of oxygen supply induces the formation of new, more fragile blood vessels. Their rupture and the microhemorrhages that follow can lead to retinal detachment. As for the wet form of AMD, it is possible to perform injections of anti-angiogenic drugs, but this treatment does not work well. Laser treatment can also be performed to burn the abnormal vessels but the effectiveness is limited.

Recently, new therapeutic approaches have been described with the aim of treating retinal diseases.

Recent therapeutic trials have explored the possibility of placing a retinal implant (artificial retina), but the resolution of this implant remains low.

Cell therapy trials have also been conducted with the aim of replacing degenerated retinal cells with stem cells capable of differentiating into epithelial, neural or vascular cells of the retina (hRPCs or human retinal progenitor cells). However, no trial has been conclusive to date, in particular cell differentiation after implantation is not controlled and intravitreal injection, which is the tested administration route, does not correspond to a physiological mechanism, which has led to undesirable side effects (https://clinicaltrials.gov/ct2/show/results/NCT02320182).

Moreover, these progenitor cells exist in vivo in humans without allowing regeneration of the adult retina (Tang et al. “Progress of stem/progenitor cell-based therapy for retinal degeneration” Journal of Translational Medicine, 10 May 2017).

In addition, membranes or sheets of retinal cells for use as implants have been described. This is the case for application US20160310637 or application EP2570139, which disclose membranes or sheets of retinal cells obtained from retinal cells taken from humans and cultured, said membranes or sheets being intended to be implanted in the eye. However, this technology is not satisfactory because it requires the production of a very large number of cells in relation to the number of grafted cells (8 batches produced to allow for quality control and the majority of cells in each batch are not positioned on the implant), moreover, implantation requires a long and complex surgery requiring specific grafting expertise for the practitioner and is very invasive for the patient.

Application EP3211071 also describes retinal tissues in the form of aggregates in suspension obtained from pluripotent stem cells. The suspension is then injected into the eye. This solution is not satisfactory either, because the injection does not make it possible to maintain a functional structuring (especially a functional polarization): i) in the case of the pigment epithelium, where the apical side of the cells must be presented facing the external segments of the photoreceptors of the graft, the survival and functionality of the transplant are limited ii) in the case of the photoreceptors, the external segment must be positioned towards the outside of the eye, opposite the cells of the pigment epithelium, and the synaptic termination must face the center of the eye to connect the rest of the neural retina. Moreover, this technique requires the injection of a large volume of liquid into the eye, and leads, as with all other solutions of the prior art, to a significant detachment of the retina, over a larger area than the area to be treated, with the risk of causing local hemorrhages during the incision or the injection. Current solutions also require three or four incisions to be made, as described in Zarbin et al. (“Concise Review: Update on Retinal Pigment Epithelium Transplantation for Age-Related Macular Degeneration” Stem Cells Translational Medicine, 2019; 8:466-477).

The objective of the invention is to overcome these various problems of the prior art, and to propose a solution for the easy, rapid and minimally invasive implantation of correctly polarized retinal cells, and their integration into the host organ, so as to replace, in a durable and assured manner, retinal cells that have degenerated in patients suffering from degenerative pathologies of the retina.

### SUMMARY OF THE INVENTION

According to the invention, the effectiveness of retinal tissue transplantation depends largely on the proper in situ incorporation (in particular, correct apical-basal polarization) of the graft via adhesion of the basal side of the pigment epithelium cells at Bruch's membrane. Therefore, to meet the objective of the invention, the inventors have developed particular hollow three-dimensional cellular arrays comprising at least one layer of retinal pigment epithelium cells organized around a cavity, said retinal pigment epithelium cells having their basal sides pointing outwards. This tissue unit preferably also comprises an outer layer of extracellular matrix on the basal side of the retinal pigment epithelium cells promoting the integration and survival of the cells, once injected into the eye. The tissue unit according to the invention may also contain other retinal cells, organized in the form of one or more layers within the retinal pigment epithelium cell layer, in the inner cavity, i.e., on the apical side of the retinal pigment epithelium cells. These other cells preferably form all or part of a retinal neural tissue.

The invention therefore relates to a hollow three-dimensional tissue unit comprising, organized around an inner cavity, at least one layer of retinal pigment epithelium cells, the basal side of each retinal pigment epithelium cell pointing outwards and the apical side pointing towards the inner cavity. The tissue unit according to the invention is preferably in the form of a hollow ovoid, a hollow cylinder, a hollow spheroid or a hollow sphere, or a section of these elements along a plane.

The invention also relates to the use of such a hollow three-dimensional tissue in the treatment of retinal diseases, in particular by implantation into the eye at Bruch's membrane. Advantageously, since the transplantation is performed with retinal tissue units according to the invention, organized and of submillimeter size, the procedure does not require the precise positioning of a single graft as in the prior art. This limits the need for (potentially traumatic) retinal detachment and consequently also the intensity of drainage of the vitreous humor from the eye. In addition, the invention has the advantage of being able to limit the surgical procedure to a single incision of the eye, as compared to three or four today.

Moreover, the polarization of the retinal pigment epithelium layer (basal side pointing outwards and apical side pointing towards the inner cavity) allows the retinal tissue units to position themselves correctly when they are transplanted into the eye and to ensure the success of the transplantation. Indeed, thanks to the positioning of their basal side on the outer side, the retinal tissue units according to the invention attach to the extracellular matrix of the back of the eye (Bruch's membrane) by emitting cells adherent to the substrate which migrate and form a monolayer.

The invention also relates to a method for preparing a hollow three-dimensional retinal tissue unit comprising the steps of:
producing a cellular microcompartment comprising, within a hydrogel capsule:
   retinal pigment epithelium cells and optionally other retinal cells, or
   cells capable of differentiating into retinal pigment epithelium cells and possibly into other retinal cells,
if the microcompartment contains cells capable of differentiating into retinal pigment epithelium cells and possibly other retinal cells: inducing cell differentiation within the cellular microcompartment so as to obtain retinal pigment epithelium cells and possibly other retinal cells,
removing the hydrogel capsules to recover the retinal pigment epithelium cells and any other retinal cells in the form of a tissue unit.

Lastly, the invention also relates to a kit for implanting hollow three-dimensional tissue units into the eye, said kit comprising at least:
between 1 and 10,000 tissue units, optionally encapsulated in hydrogel capsules, and
a surgical implantation device capable of implanting said tissue units(s) into a human eye.

### BRIEF DESCRIPTION OF THE FIGURES

FIG. 1 is a schematic representation of a cross-sectional view of a tissue unit 10 according to the invention, with a retinal pigment epithelium layer 12 and an inner cavity 14, with A corresponding to the apical side of the retinal pigment epithelium cells and B corresponding to the basal side of the retinal pigment epithelium cells.

FIG. 2 is a schematic representation of a cross-sectional view of a tissue unit 10 according to the invention, with a retinal pigment epithelium layer 12, a cavity 14 and an extracellular matrix layer 16, with A corresponding to the apical side of the retinal pigment epithelium cells and B corresponding to the basal side of the retinal pigment epithelium cells.

FIG. 3 is a schematic representation of a cross-sectional view of a microcompartment 20 comprising a hydrogel capsule 22 and a tissue unit 10 according to the invention, the tissue unit 10 being formed by a pigment epithelium layer 12, a cavity 14 and an extracellular matrix layer 16, with A corresponding to the apical side of the retinal pigment epithelium cells and B corresponding to the basal side of the retinal pigment epithelium cells.

FIG. 4 is a schematic representation of a cross-sectional view of a microcompartment 20 comprising a hydrogel capsule 22 and a tissue unit 10 according to the invention, a tissue unit 10 being formed by an extracellular matrix layer 16, a retinal pigment epithelium layer 12, a layer of retinal cells other than retinal pigment epithelium cells 18, and a cavity 14, with A corresponding to the apical side of the retinal pigment epithelium cells and B corresponding to the basal side of the retinal pigment epithelium cells.

FIG. 5 shows a microscopy image of a hydrogel (alginate) microcompartment encapsulating a tissue unit according to the invention.

FIG. 6 shows microscopy images of hydrogel (alginate) microcompartments encapsulating a small tissue unit according to the invention (A) and a large tissue unit according to the invention (B).

FIG. 7 shows microscopy images of hydrogel (alginate) microcompartments encapsulating one or more tissue units according to the invention with different cell densities.

FIG. 8 shows retinal tissue units according to the invention, without hydrogel capsule, on a substrate simulating the extracellular matrix of the fundus of the eye (here matrigel simulating Bruch's membrane) at different times until formation of a monolayer of retinal pigment epithelium.

FIG. 9 shows microscopy images and a graph showing that cell polarization can be obtained by depositing a matrix layer on the inner face of alginate capsules.

### DETAILED DESCRIPTION OF THE INVENTION

Definitions

The term “alginate” within the sense of the invention means linear polysaccharides formed from β-D-mannuronate and α-L-guluronate, salts and derivatives thereof.

The term “hydrogel capsule” within the sense of the invention means a three-dimensional structure formed from a matrix of polymer chains, swollen with a liquid and preferably water.

The term “human cells” in the sense of the invention means human cells or immunologically humanized non-human mammalian cells. Even when not specified, the cells, stem cells, progenitor cells and tissues according to the invention are formed by or obtained from human cells or from immunologically humanized non-human mammalian cells.

The term “progenitor cell” within the sense of the invention means a stem cell already engaged in cellular differentiation into retinal cells, but not yet differentiated.

The term “embryonic stem cell” within the sense of the invention means a pluripotent stem cell derived from the inner cell mass of the blastocyst. The pluripotency of embryonic stem cells can be assessed by the presence of markers such as the transcription factors OCT4 and NANOG and surface markers such as SSEA3/4, Tra-1-60 and Tra-1-81. The embryonic stem cells according to the invention are obtained without destruction of the embryo from which they are derived, for example using the technique described in Chang et al. (Cell Stem Cell, 2008, (2)): 113-117). Embryonic stem cells from human beings may potentially be excluded.

The term “pluripotent stem cell” or “pluripotent cell” within the sense of the invention means a cell that has the capacity to form all the tissues present in the entire organism of origin, without being able to form an entire organism as such. In particular, they may be induced pluripotent stem cells, embryonic stem cells or MUSE (for Multilineage-differentiating Stress Enduring) cells.

The term “induced pluripotent stem cell” within the sense of the invention means a pluripotent stem cell induced to pluripotency by genetic reprogramming of differentiated somatic cells. These cells are, in particular, positive for pluripotency markers, such as alkaline phosphatase staining and expression of the proteins NANOG, SOX2, OCT4 and SSEA3/4. Examples of processes for obtaining induced pluripotent stem cells are described in the articles Yu et al. (Science 2007, 318 (5858): 1917-1920), Takahashi et al (Cell, 207, 131(5): 861-872) and Nakagawa et al (Nat Biotechnol, 2008, 26(1): 101-106).

The term “differentiated” cells within the sense of the invention means cells that exhibit a particular phenotype, as opposed to pluripotent stem cells that are not differentiated.

The term “Feret diameter” of a tissue unit means the distance “d” between two tangents to said tissue unit, these two tangents being parallel, so that the entire projection of the tissue unit is included between these two parallel tangents.

The term “implantation” or “transplantation” into the eye within the sense of the invention means the action of depositing in the eye at a particular location at least one tissue unit according to the invention. The implantation can be carried out by any means, in particular by injection.

The term “largest dimension” of a tissue unit within the sense of the invention means the value of the largest Feret diameter of said tissue unit.

The term “smallest dimension” of a tissue unit within the sense of the invention means the value of the smallest Feret diameter of said tissue unit.

The term “tissue unit” or “retinal tissue unit” according to the invention means a unit comprising at least one tissue of the retina. The retinal tissue unit may comprise a plurality of retinal tissues assembled together with a functional structuring. The tissue unit according to the invention comprises at least one retinal pigment epithelial tissue and may also contain another retinal tissue, in particular retinal neural tissue or retinal vascular tissue, and/or at least one other constituent, for example an extracellular matrix.

Tissue Unit

The invention thereafter relates to a three-dimensional retinal tissue unit.

The tissue unit according to the invention is hollow. It always comprises an inner cavity or lumen, which constitutes the hollow part of the tissue unit. This cavity is produced at the time of formation of the tissue unit by the retinal cells that multiply and grow. The cavity contains a liquid, in particular a culture medium (such as a medium based on DMEM or DMEM-F12 and/or Neurobasal and supplemented with B27 or N-2 or NS21) and/or a liquid secreted by the cells of the tissue unit. Advantageously, the presence of this hollow portion in the retinal tissue unit allows for better integration in the retina when implanted in the eye.

The tissue unit according to the invention comprises at least one layer of retinal pigment epithelium cells. These cells are human, living cells differentiated into retinal pigment epithelium cells. The layer of retinal epithelium cells is organized around the inner cavity. The cells forming this layer together form a retinal pigment epithelium, and their basal sides all point towards the outside of the cell units, and their apical sides all point towards the inside, i.e., towards the inner cavity. The juxtaposed cells are preferably linked together on their lateral sides by tight junctions.

According to a particularly suitable embodiment, the tissue unit according to the invention also comprises an outer layer of extracellular matrix. This outer layer of extracellular matrix is located on the basal side of the retinal pigment epithelium cell. The cellular matrix layer can be formed by the cellular matrix secreted by retinal pigment epithelium cells and/or by extracellular matrix added at the time of preparation of the cell unit.

The extracellular matrix layer can form a gel. It comprises a mixture of protein and extracellular compounds necessary for the culture of the retinal pigment epithelium cells. Preferably, the extracellular matrix comprises structural proteins, such as collagen, laminins, entactin, vitronectin, and growth factors, such as TGF-beta and/or EGF. The extracellular matrix layer may consist of or comprise Matrigel® and/or Geltrex® and/or a hydrogel type matrix of plant origin, such as modified alginates, or of synthetic origin or poly(N-isopropylacrylamide) and poly(ethylene glycol)copolymer (PNIPAAm-PEG) type Mebiol®.

At the surface of the extracellular matrix layer in contact with the retinal pigment epithelium layer, the extracellular matrix may optionally contain one or more retinal pigment epithelium cells.

When the extracellular matrix is present, the retinal cells organized in three-dimensions around the inner cavity advantageously already interact with an extracellular matrix, which facilitates their implantation at the retina.

The tissue unit according to the invention may comprise one or more other layers of retinal cells other than retinal pigment epithelium cells. These cells are human cells, which are living and differentiated into retinal cells other than retinal pigment epithelium cells. The layer(s) of retinal cells other than retinal pigment epithelium cells are arranged within the retinal epithelium cell layer, i.e., on the apical side of the retinal pigment epithelium cells, organized around the lumen.

In one embodiment, the retinal cells other than retinal pigment epithelium cells are selected from rods, cones, ganglion cells, amacrine cells, bipolar cells and horizontal cells. When the cell unit comprises at least two layers of retinal cells other than retinal pigment epithelium cells, the different layers are organized successively around the inner cavity.

Preferably, the tissue unit according to the invention contains between 10 and 100,000 retinal cells.

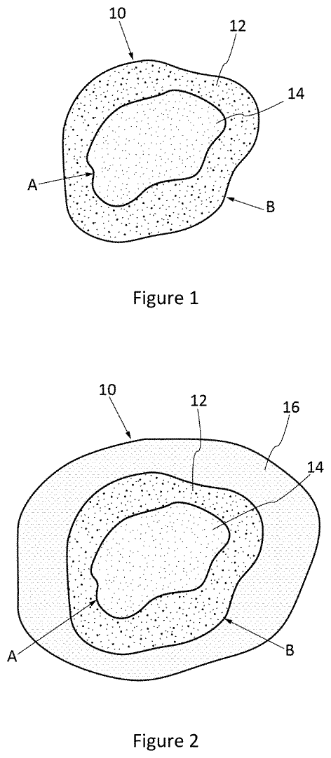

According to a particular embodiment of the invention, as shown in FIG. 1, the hollow three-dimensional retinal tissue unit 10 consists exclusively of:
an inner cavity 14, and
a layer of retinal pigment epithelium 12 organized around the inner cavity, with the basal sides B of the retinal pigment epithelial cells pointing towards the outside of the tissue unit, and the apical sides A of the retinal pigment epithelial cell pointing towards the inner cavity.

According to another particular embodiment of the invention, as shown in FIG. 2, the hollow three-dimensional retinal tissue 10 is formed exclusively of:
an inner cavity 14,
a layer of retinal pigment epithelium 12 organized around the inner cavity, with the basal sides B of the retinal pigment epithelial cells pointing towards the outside of the tissue unit, and the apical sides A of the retinal pigment epithelial cells pointing towards the inner cavity, and
a layer of extracellular matrix 16 disposed around the layer of retinal pigment epithelium cells on the basal sides of said retinal pigment epithelium cells.

The various differentiated cells constituting the tissue unit according to the invention, regardless of the embodiment, may optionally have been obtained from pluripotent stem cells, in particular human pluripotent stem cells, or optionally may have been directly reprogrammed from adult cells such as fibroblasts or peripheral blood mononuclear cells, for example.

The tissue unit according to the invention can be in any three-dimensional form, i.e., it can have the shape of any object in space. Preferably, the tissue unit according to the invention is in the form of a hollow ovoid, a hollow cylinder, a hollow spheroid or a hollow sphere. It is the outer layer of the tissue unit, i.e., the retinal pigment epithelium layer or the extracellular matrix layer when present, which confers its size and shape to the tissue unit according to the invention.

Preferably, the largest dimension of the tissue unit according to the invention is less than 1 cm, even more preferably less than 0.5 cm. According to a suitable and preferred embodiment, the largest dimension of the tissue unit according to the invention is between 100 and 1,000 μm. It may also be between 200 and 1,000 μm or between 300 and 1,000 μm. This dimension ensures easy implantation in the eye, in particular by injection, as the largest dimension should not be too large to be implanted with a prior incision of very small size. The larger the dimension, the larger the incision made in the eye and it is important to limit the size of the incision as much as possible in order to limit the risks and the impact on the treated patient. Preferably, the smallest dimension of the tissue unit is less than 1,000 μm. According to one embodiment it is between 10 and 1,000 μm, preferably between 100 and 400 μm and even more preferably between 200 and 300 μm. This smaller dimension is important for the survival of the graft in vitro, in particular to promote the survival of the retinal cells within the retinal tissue unit and to optimize the reorganization and vascularization of the tissue unit after implantation in the eye.

The thickness of the retinal pigment epithelium cell layer in the tissue unit is preferably between 5 μm and 200 μm. When the tissue unit according to the invention comprises one or more other layers of retinal cells that are not retinal pigment epithelium cells, this layer or these layers together, if there are more than one, preferably has (have) a thickness between 20 μm and 500 μm. When an outer layer of the extracellular matrix is present in the tissue unit according to the invention, the thickness of this outer layer of extracellular matrix is preferably between 30 mm and 500 mm.

The cavity preferably represents between 10% and 90% of the volume of the tissue unit according to the invention. The retinal tissue unit according to the invention is particularly useful as an implantable graft in the eye of a human being, in particular for the treatment of retinal diseases. The shape, size and constitution of the retinal cell unit according to the invention allow for the survival of the cells within the tissue unit prior to implantation and for the successful implantation, reorganization and vascularization of the graft once implanted in the eye.

Until implantation, the tissue unit may optionally be encapsulated in a hydrogel capsule, in which it has been preferentially prepared. In this case, the hydrogel capsule is preferably removed before implantation in the eye.

The tissue units according to the invention can be frozen for storage until implantation.

Implantation Kit

The invention also relates to a kit for implanting at least one tissue unit.

The implantation kit according to the invention comprises at least:
between 1 and 10,000 tissue units according to the invention, the tissue units optionally being encapsulated in a hydrogel capsule,
optionally hydrogel capsule removal means, in the case in which the capsule unit(s) are encapsulated in a hydrogel capsule,
a surgical implantation device capable of implanting said tissue unit(s) in a human eye.

The hydrogel capsule removal means must allow the capsule to be removed by hydrolysis, dissolution, piercing and/or rupture by any biocompatible means, i.e., non-toxic to the cells. The removal means are preferably selected from buffer solutions (such as phosphate buffered saline, also referred to as PBS), a buffer containing a chelator of divalent ions (such as EDTA), these being enzymes capable of lysing the hydrogel (to be selected according to the nature of the hydrogel).

The surgical implantation device can be a needle or a cannula which the internal diameter allows the passage of the tissue units according to the invention that are to be transplanted, preferably between 100 μm and 1 mm and of which the external diameter is not too traumatic for the structure of the treated eye, preferably less than 2 mm.

The number of tissue units according to the invention present in the device is between 1 and 10,000, preferably between 10 and 1,000. This number varies depending on the retinal disease to be treated and the size of the area of the retina that is no longer functional.

In the implantation kits, the tissue units can be outside the implantation device and/or already introduced in whole or in part into the surgical implantation device.

The tissue units according to the invention present in the kit can be frozen outside the device and/or frozen within the surgical implantation device. In this case, the tissue units according to the invention must be thawed prior to use by any suitable means that allows all the properties of the tissue units to be preserved. This may include, in particular, standard cell biology protocols using DMSO as antifreeze, or those applied for freezing in vitro fertilization embryos using sugars such as sucrose and alcohols such as ethylene glycol.

If the tissue units have been frozen encapsulated in a hydrogel capsule, the encapsulated tissue units should first be thawed and then the hydrogel capsules removed.

Preparation Method

The invention also relates to a method for preparing a tissue unit according to the invention. In particular, the method consists of making at least one tissue unit according to the invention by making cellular microcompartments comprising a hydrogel capsule surrounding:
differentiated retinal pigment epithelium cells and optionally other differentiated retinal cells, or
stem cells or progenitor cells capable of differentiating into retinal cells, at least into retinal pigment epithelium cells or
differentiated cells intended to undergo in the capsule:
either a trans-differentiation into retinal cells, at least into retinal pigment epithelium cells,
or a reprogramming in the capsule so that they become induced pluripotent stem cells capable of differentiating into retinal cells, at least into retinal pigment epithelium cells.

The capsule is then preferably removed so as to allow the cells of the tissue unit to implant in the retina after transplantation into the eye.

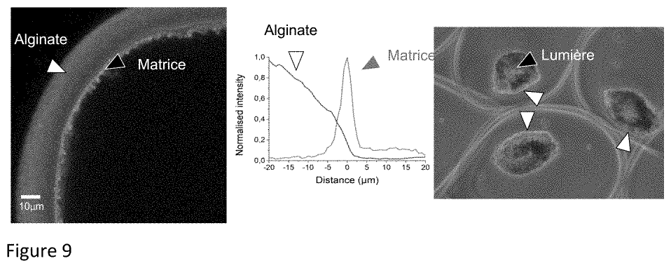

The method for preparing a tissue unit according to the invention comprises at least the implementation of the steps of:
producing a cellular microcompartment comprising, inside a hydrogel capsule:
   preferably at least elements of the extracellular matrix, secreted by the cells or provided by the operator, preferably at least part of the extracellular matrix being provided in addition to the extracellular matrix naturally secreted by the cells,
   cells capable of differentiating into at least retinal pigment epithelium cells, or at least differentiated retinal pigment epithelium cells,
if the cells introduced into the microcompartment are cells capable of differentiating into at least retinal pigment epithelium cells: inducing cell differentiation within the cellular microcompartment, so as to obtain at least retinal pigment epithelium cells and possibly other retinal cells,
removing the hydrogel capsules to recover the retinal pigment epithelium cells and possibly other retinal cells in the form of a hollow three-dimensional retinal tissue comprising, organized around an inner cavity, at least one retinal pigment epithelium layer, the basal side of each retinal pigment epithelium cell of which points outwards and the apical side towards the cavity. Advantageously, the total or partial encapsulation in the hydrogel and the provision of extracellular matrix combined is a means capable of allowing the polarization of the retinal pigment epithelium cells. Indeed, the polarization of said cells can be obtained by depositing a layer of matrix on the inner face of the hydrogel capsules which positions the basal side of the cells, the tissue organizes itself around the cavity following this indication of polarity (as illustrated in FIG. 9, which shows that an extracellular matrix layer anchored to the alginate shell induces polarization of the cells as evidenced by the flattening of the tissue against the alginate due to the high tensile strength of the gel dictating the shape of the tissue). The used hydrogel is preferably biocompatible, i.e., not toxic to cells. The hydrogel capsule must allow the diffusion of oxygen and of nutrients to feed the cells contained in the microcompartment and allow their survival. According to one embodiment, the capsule comprises alginate. It can be formed exclusively of alginate. In particular, the alginate may be a sodium alginate, composed of 80% α-L-guluronate and 20% β-D-mannuronate, with an average molecular weight of 100 to 400 kDa and a total concentration between 0.5 and 5% by weight.

The hydrogel capsule makes it possible to protect the cells from the external environment, to limit the uncontrolled proliferation of the cells, and allows for controlled differentiation of the cells into retinal cells, at least into retinal pigment epithelium cells. A capsule very preferably surrounds a single tissue unit according to the invention and each tissue unit is surrounded by a single hydrogel capsule.

Once the retinal tissue unit according to the invention is obtained, i.e., when the cells are differentiated into retinal cells including at least one layer of retinal pigment epithelium cells, and the shape and size are as desired, the capsule is removed. Removal of the capsule can be performed at the end of the method or later in time before implantation in the eye. Removal of the capsule can be achieved in particular by hydrolysis, dissolution, piercing and/or rupture by any means that is biocompatible i.e., non-toxic to the cells. For example, removal can be achieved using a phosphate buffered saline, a chelator of divalent ions, an enzyme such as alginate lyase if the hydrogel comprises alginate and/or laser microdissection. Since the removal of the hydrogel is complete, the tissue unit according to the invention is hydrogel-free when implanted in the eye.

Any method for producing cellular microcompartments containing within a hydrogel capsule at least retinal pigment epithelium cells or cells capable of yielding at least retinal pigment epithelium cells and optionally extracellular matrix and/or retinal cells other than retinal pigment epithelium cells or cells capable of yielding at least retinal cells other than retinal pigment epithelium cells can be used. A suitable method is described in particular in application WO2018/096277.

In a particular embodiment, the step of producing a cellular microcompartment of the preparation method according to the invention comprises the steps of:
incubating pluripotent stem cells in a culture medium, preferably a culture medium based on DMEM or DMEM-F12, FGF-2 or a molecule replicating its action on the cell, TGF-beta or a molecule replicating its action on the cell,
mixing the pluripotent stem cells with an extracellular matrix,
encapsulating the mixture in a hydrogel layer.

The encapsulated cells for the preparation of a tissue unit according to the invention are preferably selected from:
cells capable of differentiating into at least retinal pigment epithelium cells, these cells being:
   stem cells capable of differentiating into retinal cells, at least retinal pigment epithelium cells, preferably embryonic stem cells or induced pluripotent stem cells, very preferably induced pluripotent stem cells, and/or
   progenitor cells capable of differentiating into retinal cells, at least into retinal pigment epithelium cells,
and/or differentiated retinal pigment epithelium cells and possibly differentiated retinal cells other than retinal pigment epithelium cells,
and/or differentiated cells capable of undergoing trans-differentiation into retinal cells, at least into retinal pigment epithelium cells,
and/or differentiated cells capable of undergoing reprogramming so as to become induced pluripotent stem cells capable of differentiating into retinal cells, at least into retinal pigment epithelium cells.

The encapsulated cells may be immunocompatible with the person intended to receive the tissue unit, to avoid any risk of rejection. In one embodiment, the encapsulated cells have been previously harvested from the person into whom the one or more tissue units are to be implanted.

Differentiation into retinal pigment epithelium cells can be achieved by any suitable process. This may include a known method, such as one of the methods described in Leach et al. (“Concise Review: Making Stem Cells Retinal: Methods for Deriving Retinal Pigment Epithelium and Implications for Patients With Ocular Disease”, Stem Cells 2015; 33:2363-2373). The basal medium can be DMEM (“Dulbecco's modified Eagle's medium”) or DMEMF12, which can be supplemented with KSR-XF (“KnockOut DMEM medium”) or N-2 and/or B27, 1% GlutaMax, and 1% non-essential amino acid solution. Induction can also be achieved with the sequence as published in Choudhary et al. (“Directing Differentiation of Pluripotent Stem Cells Toward Retinal Pigment Epithelium Lineage” STEM CELLS TRANSLATIONAL MEDICINE 2016; 5:1-12).

Differentiation into retinal cells other than retinal pigment epithelium cells can be achieved by any suitable method. This may include a method such as the method described in Barnea-Cramer et al. (“Function of human pluripotent stem cell-derived photoreceptor progenitors in blind mice” Nature, Scientific Reports, published 13 Jul. 2016). Differentiation can thus be performed with DMEMF12, which can be supplemented with KSR-XF (KnockOut DMEM medium) or N-2 and/or B27, 1% GlutaMax, and 1% non-essential amino acid solution.

In a particular embodiment, the cell differentiation induction step of the preparation method according to the invention comprises the steps of:
growing the microcompartment in a pluripotent cell culture medium until at least 10 cells are obtained, preferably 100 cells,
growing the microcompartment in a DMEMF12 culture medium supplemented with N-2 and B27, 1% GlutaMax, and 1% non-essential amino acid solution and LDN193189 and 5B431542 and 20 μg/ml of human insulin,
growing the microcompartment in a DMEMF12 culture medium supplemented with N-2 and B27, 1% GlutaMax, and 1% non-essential amino acid solution and LDN193189 and 5B431542,
growing the microcompartment in a DMEMF12 culture medium supplemented with N-2 and B27 1% GlutaMax, and 1% non-essential amino acid solution and 10 ng/ml of human BDNF, 10 ng/ml of human CNTF, 2 μM of retinoic acid and 10 μM of DAPT.

The microcompartments are preferably grown for at least 18 days, preferably between 18 and 50 days.

In an embodiment of a tissue unit comprising a retinal pigment epithelium layer and at least one layer of other retinal cells, the method consists of co-encapsulating retinal pigment epithelium cells and the other cells. The cells obtained from a few days of each differentiation are preferably co-encapsulated to form a structure containing a retinal pigment epithelium and a neural retina. The cells are then matured in the capsule for 5 to 50 days, more preferably 10 to 25 days, before obtaining a tissue unit according to the invention.

The cavity is produced at the time of formation of the three-dimensional tissue unit, by the cells multiplying and growing.

The cavity may contain a liquid and in particular the culture medium used for carrying out the method.

The method according to the invention may include a step of amplifying the retinal pigment epithelium cells, in the microcompartment.

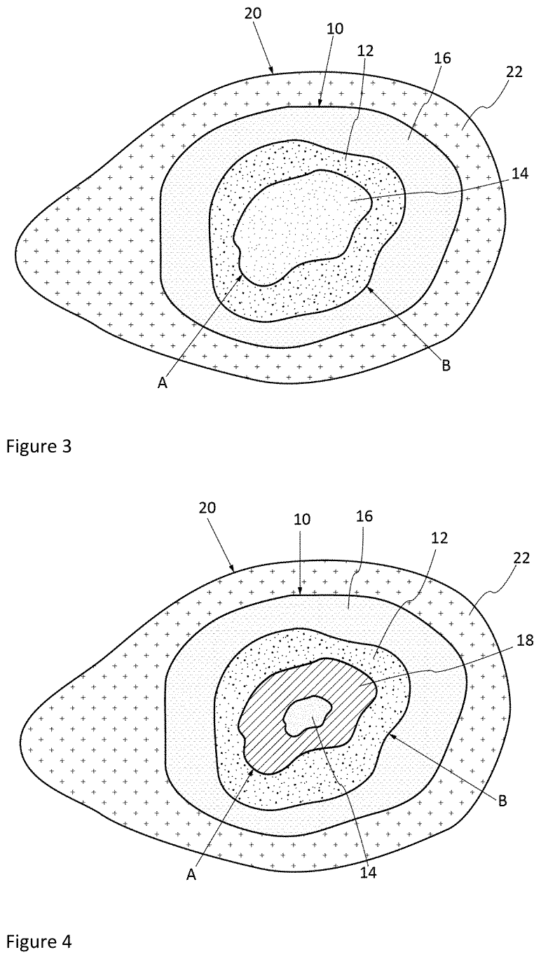

An embodiment of a microcompartment 20 comprising a hollow three-dimensional tissue unit 10 according to the invention is shown in FIG. 3. In this embodiment, the microcompartment is formed exclusively of:
a hydrogel layer 22, and
a tissue unit 10, formed of:
   an inner cavity 14,
   a layer of retinal pigment epithelium 12 organized around the inner cavity, with the basal sides B of the retinal pigment epithelial cells pointing towards the outside of the tissue unit, and the apical sides A of the retinal pigment epithelial cells pointing towards the inner cavity.
   a layer of extracellular matrix 16 arranged around the layer of retinal pigment epithelium cells, on the basal sides of said retinal pigment epithelium cells.

Another embodiment of a microcompartment 20 comprising a hollow three-dimensional retinal tissue unit according to the invention is shown in FIG. 4. In this embodiment, the microcompartment consists exclusively of:
a hydrogel layer 22, and
a tissue unit 10, formed of:
   an inner cavity 14,
   a layer 18 of retinal cells other than retinal pigment epithelial cells, preferably a layer of neural retina,
   a layer of retinal pigment epithelium 12 organized around the inner cavity, the basal sides B of the retinal pigment epithelial cells pointing towards the outside of the tissue unit, and the apical sides A of the retinal pigment epithelial cells pointing towards the inner cavity,
   a layer of extracellular matrix 16 arranged around the layer of retinal pigment epithelium cells on the basal sides of said retinal pigment epithelium cells.

After the differentiation step, at any time prior to implantation of the tissue units into the eye, the method according to the invention may include a step of verifying the phenotype of the cells contained in the capsule. This verification can be performed by identifying the expression of RPE65 by the pigment epithelium in the outer position of the tissue units, in a certain embodiment and in particular for the case of tissue elements containing neural retina elements of recoverin in the inner position of the tissue units, at the cavity, expressed by all photoreceptors and rhodopsin/PDE6beta for rods, in a certain embodiment the lumen can contain vitrosin and opticin.

The method according to the invention may comprise a step of freezing the microcompartments containing the tissue units according to the invention before removal of the hydrogel layer or freezing the tissue units after the step of hydrogel capsule removal. The freezing is preferably performed at a temperature between −190° C. and −80° C.

The microcompartments containing the tissue units according to the invention before removal of the hydrogel layer or the tissue units after the step of removal of the hydrogel capsule can be stored under the following conditions between +4° C. and room temperature. The tissue units according to the invention can also be used directly after carrying out the method according to the invention, without storage.

The preparation method according to the invention, before or after possible thawing of the microcompartments containing the tissue units prior to removal of the hydrogel layer or the tissue units, may also comprise an additional step of loading a surgical implantation device with at least one tissue unit according to the invention, preferably between 10 and 1,000 tissue units, even more preferably between 10 and 100 tissue units.

Implantation of Tissue Units in the Eye

The invention also relates to a hollow three-dimensional tissue unit comprising, organized around an inner cavity, at least one layer of retinal pigment epithelium cells, with the basal side of each retinal pigment epithelium cell pointing towards the outside and the apical side pointing towards the inner cavity, for use in the treatment of a retinal disease, in particular in a patient in need thereof. The term “treatment” means a preventative, curative or symptomatic treatment, i.e., any act intended to improve a person's sight temporarily or permanently, and preferably also to eradicate the disease and/or to stop or delay the progression of the disease and/or to promote the regression of the disease.

Indeed, the tissue units according to the invention can be used for the treatment of retinal diseases in humans, in particular degenerative retinal diseases, and preferably a disease selected from age-related macular degeneration, diabetic retinopathy, retinopathies related to trauma to the eye and hereditary retinopathies.

The treatment consists of implanting, that is to say transplanting the tissue units according to the invention into the eye, at the retina, and in particular at Bruch's membrane, i.e., between Bruch's membrane and the neural retina. A surgical implantation device suitable for implantation in the eye is very preferably used. This may include, in particular, needles, cannulas or other devices for depositing the tissue units, such as those used for the implantation of stents in arteries or surgical micro implants. Implantation can be performed in particular by carrying out the steps consisting of:
penetrating or making an incision in the retina using the surgical implantation device in the treatment area,
injecting the tissue units under the retina, preferably at Bruch's membrane, i.e., between Bruch's membrane and the neural retina,
removing the surgical implant device, preventing the cells from being pushed back into the vitreous humor.

In an embodiment, during a single implantation, between 1 and 10,000 tissue units according to the invention are implanted. If necessary, it is possible to carry out several simultaneous or successive implantations in different areas of the retina, preferably at Bruch's membrane, in particular in the case in which several separate areas are affected by the disease or if the area where the transplant is to be performed is too extensive to perform a transplant in only one place.

Similarly, if a single transplant is not sufficient in one area, several implantations can be performed repeatedly in the same area over a shorter or longer period of time.

The implantation of tissue units according to the invention allows patients suffering from retinal diseases, and in particular degenerative retinal diseases, to regain at least partial sight.

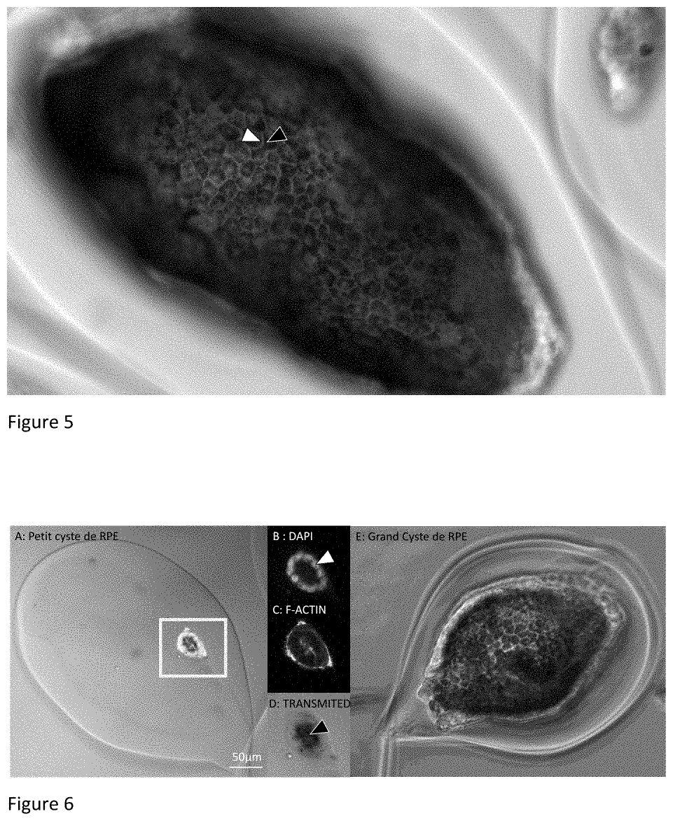

The invention will now be illustrated by results shown in FIGS. 5 to 8.

The retinal epithelium tissues illustrated in these various figures were obtained by implementing a method comprising the steps of:
producing a cellular microcompartment comprising, within a hydrogel capsule:
   extracellular matrix elements provided by the operator,
   retinal pigment epithelium cells,

These retinal pigment epithelium cells can be obtained in the following way:
growing induced pluripotent stem cells within i) a petri dish until colonies containing several tens of cells are obtained ii) the microcompartment in a pluripotent cell culture medium, until at least 10 cells, preferably 100 cells, are obtained in the microcompartment,
growing: i) colonies - ii) the microcompartment in a DMEMF12 culture medium supplemented with N-2 and B27, 1% GlutaMax, and 1% non-essential amino acid solution and LDN193189 and SB431542 and 20 μg/ml of human insulin,
growing: i) colonies ii) the microcompartment in a DMEMF12 culture medium supplemented with N-2 and B27, 1% GlutaMax, and 1% non-essential amino acid solution and LDN193189 and SB431542, growing: i) the colonies ii) the microcompartment in a DMEMF12 culture medium supplemented with N-2 and B27, 1% GlutaMax, and 1% non-essential amino acid solution and 10 ng/ml of human BDNF 10 ng/ml of human CNTF, 2 μM of retinoic acid and 10 μM of DAPT. In FIG. 5, the capsule obtained by encapsulation of retinal pigment epithelial cells derived from the differentiation of the induced cells proliferated within the microcompartment to organize itself in the form of a polarized tissue unit. FIG. 5 shows clearly the formation of a mature squamous pigmented monostratified epithelium (black arrow) (typically enabled by the formation of tight junctions (white arrow)) typical of the tissue structure of retinal pigment epithelial cells in vivo.

In FIG. 6, the capsules obtained by encapsulation of retinal pigment epithelial cells derived from differentiation of induced pluripotent cells show the pigmented epithelial sheet structuring regardless of the number of cells in the tissue (approximately 100 cells on the left, size: 50 μm, insert A, and approximately 1,000 cells on the right, size: 250 μm, insert B) typical of the tissue structure of retinal pigment epithelial cells in vivo. More particularly, it can be seen in FIG. 6 that the cells are organized in a hollow spheroid (insert B) and a hollow ovoid (insert E) according to an apical-basal polarization characterized in that the apical side points towards the inside of the tissue unit and the basal side towards the outside of the tissue unit located here in contact with the alginate layer. In particular, the extracellular pigments are located on the apical side of the cells (insert D, transmitted light, white arrow) with a basal organization of the nuclei (insert B, DAPI, white arrow) corresponding to the topology encountered in vivo.

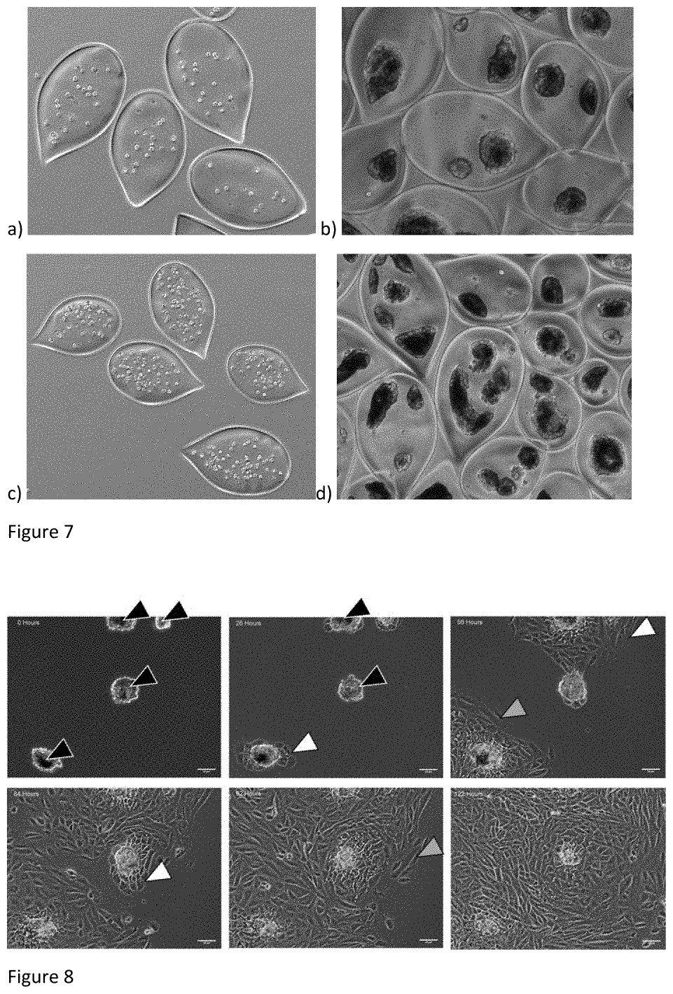

In FIG. 7, the capsules obtained by encapsulation of retinal pigment epithelial cells derived from the differentiation of induced pluripotent cells were amplified 10-fold in 4 weeks (FIGS. 7a and 7b) and 4-fold in 4 weeks (FIGS. 7c and 7d).

These figures illustrate that the retinal tissue units according to the invention can have variable cell densities.

Lastly, in the sequential images of FIG. 8, several retinal tissue units according to the invention (without hydrogel capsule) have been arranged on a substrate simulating the extracellular matrix of the fundus (here, matrigel simulating Bruch's membrane). It can be seen that the retinal tissue units (black arrows) are able, thanks to the positioning of their basal side on the outside, to attach to the substrate simulating the extracellular matrix of the fundus by emitting cells adherent to the substrate (white arrows) which migrate (gray arrows) to cover the substrate and form a monolayer (insert at the bottom right corresponding to 122 hours of culture).

1. A hollow three-dimensional retinal tissue unit comprising, organized around an inner cavity, at least one layer of differentiated living human retinal pigment epithelium cells, with the basal side of each retinal pigment epithelium cell pointing outwards and the apical side pointing towards the inner cavity.

2. The retinal tissue unit according to claim 1, characterized in that it also comprises an outer layer of extracellular matrix located on the basal side of the retinal pigment epithelium cells.

3. The retinal tissue unit of claim 1, characterized in that it is in the form of a hollow ovoid, a hollow cylinder, a hollow spheroid or a hollow sphere.

4. The retinal tissue unit of claim 3, characterized in that its largest dimension is between 100 and 1,000 μm.

5. The retinal tissue unit of claim 4, characterized in that its smallest dimension is between 10 and 1,000 μm.

6. The retinal tissue unit of claim 1, characterized in that the juxtaposed retinal pigment epithelium cells are connected to one another on their lateral sides by tight junctions.

7. The retinal tissue unit of claim 1, characterized in that it also comprises, on the apical side of the retinal pigment epithelium cells, organized around the inner cavity, at least one layer of differentiated living human retinal cells other than retinal pigment epithelium cells.

8. The retinal tissue unit of claim 7, characterized in that the differentiated living human retinal cells other than retinal pigment epithelium cells are selected from rods, cones, ganglion cells, amacrine cells, bipolar cells and horizontal cells.

9. The retinal tissue unit of claim 1, characterized in that it contains from 10 to 100,000 retinal cells.

10. The retinal tissue unit of claim 1, characterized in that the retinal pigment epithelium cells and/or any other retinal cells were obtained from induced pluripotent stem cells (IPS).

11. The retinal tissue unit of claim 1, characterized in that it is encapsulated in a hydrogel capsule.

12. The retinal tissue of claim 1, for use in the treatment of a retinal disease.

13. A retinal tissue unit for use according to claim 12, in the treatment of a degenerative retinal disease.

14. A retinal tissue unit for use according to claim 13 in the treatment of a retinal disease selected from age-related macular degeneration, diabetic retinopathy, trauma-related retinopathies of the eye and hereditary retinopathies.

15. A method for preparing a retinal tissue unit according to claim 1, comprising the steps of:
producing a cellular microcompathnent comprising, within a hydrogel capsule:
optionally at least extracellular matrix elements, secreted by the cells or added,
cells capable of differentiating into at least retinal pigment epithelium cells or at least differentiated retinal pigment epithelium cells,
if the cells introduced into the microcomparment are cells capable of differentiating into at least retinal pigment epithelium cells: inducing cell differentiation within the cellular microcomparment, so as to obtain at least retinal pigment epithelium cells and possibly other retinal cells,
removing the hydrogel capsules in order to recover the retinal pigment epithelium cells and any other retinal cells in the form of a hollow three-dimensional retinal tissue unit.

16. The method according to claim 15, characterized in that it comprises a step of amplifying the retinal pigment epithelium cells.

17. The method according to claim 15, characterized in that the cells capable of differentiating into at least retinal pigment epithelium cells are pluripotent stem cells.

18. The method according to claim 17, characterized in that the pluripotent stem cells are induced pluripotent stem cells (IPS).

19. The method for preparing a retinal tissue unit according to claim 15, characterized in that it comprises a further step of loading said tissue unit into a surgical implantation device suitable for injection into the eye.

20. A kit for implanting tissue units according to claim 1 into the eye, characterized in that the kit comprises:
between 1 and 10,000 tissue units according to claim 1,
a surgical implantation device capable of implanting said tissue unit(s) into a human eye.

21. A kit for implanting tissue units according to claim 11 into the eye, characterized in that the kit comprises:
between 1 and 10,000 tissue units according to claim 11,
hydrogel capsule removal means,
a surgical implantation device capable of implanting said tissue unit(s) into a human eye.
